SEQUENCE LISTING (33 sequences)

SEQ ID NO: 1 - protein, 71 aa, Artificial Sequence
  v-MIPII derived peptide
LGASWHRPDKCCLGYQKRPLPQVLLSSWYPTSQLCSKPGVIFLTKRGRQVCADKSKDWVKKLMQQLPVTAR

SEQ ID NO: 2 - protein, 21 aa, Artificial Sequence
  v-MIPII derived peptide
LGASWHRPDKCCLGYQKRPLP

SEQ ID NO: 3 - protein, 13 aa, Artificial Sequence
  v-MIPII derived peptide
HRPDKCCLGYQKR

SEQ ID NO: 4 - protein, 10 aa, Artificial Sequence
  v-MIPII derived peptide
LGASWHRPDK

SEQ ID NO: 5 - protein, 22 aa, Artificial Sequence
  v-MIPII derived peptide
LGYQKRPLPQVLLSSWYPTSQL

SEQ ID NO: 6 - protein, 20 aa, Artificial Sequence
  v-MIPII derived peptide
KPVSHRPDKCCLGYQKRPLP

SEQ ID NO: 7 - protein, 23 aa, Artificial Sequence
  v-MIPII derived peptide
QVLLSSWYPTSQLCSKPGVIFLT

SEQ ID NO: 8 - protein, 22 aa, Artificial Sequence
  v-MIPII derived peptide
SKPGVIFLTKRGRQVCADKSKD

SEQ ID NO: 9 - protein, 20 aa, Artificial Sequence
  v-MIPII derived peptide
ADKSKDWVKKLMQQLPVTAR

SEQ ID NO: 10 - protein, 11 aa, Artificial Sequence
  v-MIPII derived peptide
CTSQLASKPGC

SEQ ID NO: 11 - protein, 11 aa, Artificial Sequence
  v-MIPII derived peptide
CFLTKRGRQVC

SEQ ID NO: 12 - protein, 21 aa, Artificial Sequence
  v-MIPII derived peptide
LGASWHRPDKAALGYQKRPLP

SEQ ID NO: 13 - protein, 21 aa, Artificial Sequence
  v-MIPII derived peptide
AGASWHRPDKCCLGYQKRPLP

SEQ ID NO: 14 - protein, 21 aa, Artificial Sequence
  v-MIPII derived peptide
LGASAHRPDKCCLGYQKRPLP

SEQ ID NO: 15 - protein, 21 aa, Artificial Sequence
  v-MIPII derived peptide
LGASWHAPDKCCLGYQKRPLP

SEQ ID NO: 16 - protein, 21 aa, Artificial Sequence
  v-MIPII derived peptide
LGASWHRPDACCLGYQKRPLP

SEQ ID NO: 17 - protein, 21 aa, Artificial Sequence
  v-MIPII derived peptide
LGASWHRPDKACLGYQKRPLP

SEQ ID NO: 18 - protein, 21 aa, Artificial Sequence
  v-MIPII derived peptide
LGASWHRPDKCCLGYAKRPLP

SEQ ID NO: 19 - protein, 21 aa, Artificial Sequence
  v-MIPII derived peptide
LGASWHRPDKCCLGYQKAPLP

SEQ ID NO: 20 - protein, 21 aa, Artificial Sequence
  v-MIPII derived peptide
PLPRKQYGLCCKDPRHWSAGL

SEQ ID NO: 21 - protein, 21 aa, Artificial Sequence
  v-MIPII derived peptide
LGASWHRPDKCCLGYQKRPLP

SEQ ID NO: 22 - protein, 21 aa, Artificial Sequence
  v-MIPII derived peptide
PLPRKQYGLCCKDPRHWSAGL

SEQ ID NO: 23 - protein, 21 aa, Artificial Sequence
  v-MIPII derived peptide
LGASWHRPDKCCLGYQKRPLP

SEQ ID NO: 24 - protein, 21 aa, Artificial Sequence
  v-MIPII derived peptide
LGASWHRPDKCCLGYQKRPLP

SEQ ID NO: 25 - protein, 21 aa, Artificial Sequence
  v-MIPII derived peptide
AGASWHRPDKCCLGYQKRPLP

SEQ ID NO: 26 - protein, 21 aa, Artificial Sequence
  v-MIPII derived peptide
LGASAHRPDKCCLGYQKRPLP

SEQ ID NO: 27 - protein, 21 aa, Artificial Sequence
  v-MIPII derived peptide
LGASWHAPDKCCLGYQKRPLP

SEQ ID NO: 28 - protein, 21 aa, Artificial Sequence
  v-MIPII derived peptide
LGASWHRPDACCLGYQKRPLP

SEQ ID NO: 29 - protein, 21 aa, Artificial Sequence
  v-MIPII derived peptide
LGASWHRPDKACLGYQKRPLP

SEQ ID NO: 30 - protein, 21 aa, Artificial Sequence
  v-MIPII derived peptide
LGASWHRPDKCCLGYAKRPLP

SEQ ID NO: 31 - protein, 21 aa, Artificial Sequence
  v-MIPII derived peptide
LGASWHRPDKCCLGYQKAPLP

SEQ ID NO: 32 - protein, 21 aa, Artificial Sequence
  v-MIPII derived peptide
LGASWHRPDKAALGYQKRPLP

SEQ ID NO: 33 - protein, 10 aa, Artificial Sequence
  v-MIPII derived peptide
LGASWHRPDK

(19) United States
(12) Patent Application Publication
(10) Pub. No.: US 2003/0220482 A1
(43) Pub. Date: Nov. 27, 2003

(54) Novel peptide antagonist of CXCR4 derived from the N-terminus of the viral chemokine vMIP-II

(76) Inventors: Ziwei Huang, (US); Jiansong Luo, (US); Zhaowen Luo, (US); Naming Zhou, (US)

Correspondence Address: DRINKER BIDDLE & REATH, ONE LOGAN SQUARE, 18TH AND CHERRY STREETS, PHILADELPHIA, PA, 19103-6996, US

(21) Appl. No.: 09/773,830

(22) Filed: Feb. 1, 2001

Related U.S. Application Data
(60) Provisional application No. 60/180,487, filed on Feb. 3, 2000.

(51) Int. Cl. C07K 14/52

(52) U.S. Cl. 530/351; 530/326

(57) ABSTRACT

The viral Macrophage Inflammatory Protein-II (vMIP-II) is a chemokine that interacts with the CC and CXC chemokine receptors, including the CCR5 and CXCR4 chemokine receptors. CCR5 and CXCR4 are the principal coreceptors required for cell entry of human immunodeficiency virus type 1 (HIV-1). The present invention describes a peptide fragment of the vMIP-II that prevents the HIV-1 virus from interacting with the coreceptor CXCR4, thereby preventing viral infection of that cell. These peptide fragments will serve as lead compounds for the development of therapeutic agents against HIV-1 infections.

### CROSS REFERENCE TO RELATED APPLICATIONS

This application claims priority under 35 U.S.C. 119 based upon U.S. Provisional Application No. 60/180,487 filed Feb. 3, 2000.

### FIELD OF THE INVENTION

The present invention relates to the field of molecular biology, more particularly to the binding of the viral Macrophage Inflammatory Protein-II (vMIP-II), and fragments thereof, to chemokine receptors, thereby inhibiting entry of human immunodeficiency virus (HIV-1) into target cells.

### BACKGROUND OF INVENTION

Chemokines are a superfamily of small proteins of pro-inflammatory mediators and potent chemoattractants for T cells, monocytes and macrophages. Based on the positions of two conserved cysteine residues in their N-termini, chemokines can be mainly divided into CC and CXC subfamilies (Wells, T. N. C., et al., J Leuk Biol, 59:53-60, 1996). Chemokine receptors play an important role as coreceptors for the entry of HIV-1 into the target cell, among which CCR5 and CXCR4 are the two major HIV-1 coreceptors (Broder, C. and Berger, E., Proc Natl Acad Sci USA, 92:9004-9008, 1995). Human CC chemokines such as RANTES and MIP-1β (Cocchi, F., et al., Science, 270:1811-1815) and CXC chemokines such as SDF-1β (Bleul, C. C., et al., Nature, 382:829-833, 1996; Oberlin, E., et al, Nature, 382:833-835, 1996) inhibit HIV-1 entry via CCR5 and CXCR4 receptors, respectively. In general, a particular chemokine can only bind one or more receptors within the same subfamily. However, viral Macrophage Inflammatory Protein-II (vMIP-II), a chemokine encoded by human herpesvirus 8 (HHV-8) (Moore, P. S., et al., Science, 274:1739-1744, 1996), displays diverse interactions with both CC and CXC chemokine receptors and inhibits HIV-1 entry mediated through CCR3, CCR5, and CXCR4 (Boshoff, C., et al., Science, 278:290-294, 1997; Kledal, T. N., et al., Science, 277:1656-1659, 1997). The broad-spectrum receptor binding property of vMIP-II is unique among all known chemokines and thus provides a useful template to study chemokine ligand-receptor interaction and design novel small molecule anti-HIV agents. An important question regarding the mechanism of action of vMIP-II is whether it uses common regions for the general binding of multiple receptors or if distinctive sites within vMIP-II have evolved for the selective interaction with different receptors.

In the present invention, a synthetic peptide approach to probe the mechanism of the biological function of vMIP-II is described. The comparison of amino acid sequences of vMIP-II (SEQ. ID. NO: 1) and other human chemokines reveals that the N-terminus of vMIP-II has little homology with either CC or CXC chemokines, whereas other regions of vMIP-II share a high sequence similarity with CC chemokines, such as MIP-1α and MIP-1β (Kledal, T. N., et al., Science, 277:1656-1659, 1997). It is known that the N-termini in a number of other chemokines are critical for biological function (Clark-Lewis, I., et al., J Leuk Biol, 57:703-711, 1995). Therefore, it is conceivable that the unique N-terminal sequence of vMIP-II confers a biological function that is distinct from other chemokines. In the present invention, a synthetic peptide derived from the N-terminus of vMIP-II was synthesized and studied in various biological assays. The peptide, designated as V1, contains the amino acid sequence of residues 1-21 of vMIP-II (LGASWHRPDKCCLGYQKRPLP, SEQ. ID. NO: 2). This peptide displayed antagonistic activity against CXCR4, but not CCR5, and selectively inhibited CXCR4-mediated T- and dual-tropic HIV entry. The present invention describes the functional determinants of vMIP-II that are required for interactions with chemokine receptors. In addition, these functional determinants will serve as lead compounds in the development of novel anti-HIV agents.

### SUMMARY OF THE INVENTION

It is an object of the present invention that a peptide fragment of a viral Macrophage Inflammatory Protein-II (vMIP-II) selectively prevents CXCR4 signal transduction and coreceptor function in mediating the entry of HIV-1. It is a further object that this peptide fragment be a fragment of the amino-terminal end of the vMIP-II. More particularly, residues 1-21 (SEQ ID NO: 2), or any subfragments therein, of the vMIP-II. It is a further object of the present invention that this peptide fragment serve as a lead compound for the development of novel small molecular agents to prevent HIV-1 from entering a cell.

It is another object of the present invention for a peptide of the formula X—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—Y. to have the following amino acids: where X is a substituent attached on the N-terminal of a peptide, X can be H, CH₃CO, C₆H₅CO, or C₆H₅CH₂CO; Y is a substituent attached on the C-terminal of a peptide with the following general structure, C(α)-CO—Y

Y can be OH, NH₂, OCH₃, OCH₂C₆H₅, or NHCH₃; Y can be from zero to nine amino acids,

R₁ is Ile, Leu, Val, or Phe;

R₂ is Gly, Ala;

R₃ is Ala, Gly;

R₄ is Ser, Thr, or Tyr;

R₅ is Trp, Phe, Tyr;

R₆ is His, Lys, Arg, or Tyr;

R₇ is Arg, His, or Lys;

R₈ is Pro, Leu, or Val;

R₉ is Asp, Glu, Arg, or Lys;

R₁₀ is Lys, Arg, or His;

R₁₁ is Cys, Ser, or Ala;

R₁₂ is Cys, Ser, or Ala;

R₁₃ is Ile, Leu, or Val;

R₁₄ is Gly, Ala;

R₁₅ is Tyr, Thr, Ser;

R₁₆ is Gln, Asn, Arg, or Lys;

R₁₇ is Lys, Arg, or His;

R₁₈ is Arg, His, or Lys;

R₁₉ is Pro, Leu, or Val;

R₂₀ is Ile, Leu, or Val;

R₂₁ is Pro, Leu, or Val;

and if R₁₁ is Cys then R₁₂ can be Cys, penicillamine or tertiary butyloxycarbonyl-a-aminobutyric acid; if R₁₂ is Cys then R₁₁ can be Cys, penicillamine, tertiary butyloxycarbonyl-a-aminobutyric acid, and, R₁₁ and R₁₂ can be penicillamine, or tertiary butyloxycarbonyl-a-aminobutyric acid; and, R₁₁ and R₁₂ can be Ala.

It is a further object of the present invention for a preferred embodiment, to have the following formula: X can be H, or CH₃CO; Y can be OH, or NH₂; and, R₁ is Leu, R₂ is Gly, R₃ is Ala, R₄ is Ser, R₅ is Trp, R₆ is His, R₇ is Arg, R₈ is Pro, R₉ is Asp, R₁₀ is Lys, R₁₁ is Cys, R₁₂ is Cys, R₁₃ is Leu, R₁₄ is Gly, R₁₅ is Tyr, R₁₆ is Gln, R₁₇ is Lys, R₁₈ is Arg, R₁₉ is Pro, R₂₀ is Leu, R₂₁ is Pro.

The present invention has a most preferred embodiment, for the peptide, which is X is H, Y is NH₂; and, R₁ is Leu, R₂ is Gly, R₃ is Ala, R₄ is Ser, R₅ is Trp, R₆ is His, R₇ is Arg, R₈ is Pro, R₉ is Asp, R₁₀ is Lys, R₁₁ is Cys, R₁₂ is Cys, R₁₃ is Leu, R₁₄ is Gly, R₁₅ is Tyr, R₁₆ is Gln, R₁₇ is Lys, R₁₈ is Arg, R₁₉ is Pro, R₂₀ is Leu, R₂₁ is Pro.

It is another object of the present invention that a preferred embodiment have a C-terminal truncation peptide containing at least the following fragment:

X—R₁—R₂—R₃—R₄—R₅—R₆—R₇—R₈—Y,

and where:

R₁ is Ile, Leu, or Phe;

R₂ is Gly, Ala, or Val;

R₃ is Ala, Val, or Gly;

R₄ is Ser, Thr, or Tyr;

R₅ is Trp, Phe, Tyr, or Leu;

R₆ is His, Lys, Arg, or Trp;

R₇ is Arg, His, or Lys;

R₈ is Pro, Leu, or Val.

and, a C-terminal truncation peptide preferably containing at least a following fragment, wherein X is H, Y is NH₂; and, R₁ is Leu, R₂ is Gly, R₃ is Ala, R₄ is Ser, R₅ is Trp, R₆ is His, R₇ is Arg, R₈ is Pro, R₉ is Asp, R₁₀ is Lys. It is a further object of the present invention for the peptide to be between 3-30 amino acids, preferably 8-21 amino acids.

In another embodiment of the present invention a synthetic peptide's amino acids are D amino acids, having the formula: X—R_{1d}—R_{2d}—R_{3d}—R_{4d}—R_{5d}—R_{6d}—R_{7d}—R_{8d}—R_{9d}—R_{10d}—R_{11d}—R_{12d}—R_{13d}—R_{14d}—R_{15d}—R_{16d}—R_{17d}—R_{18d}—R_{19d}—R_{20d}—R_{21d}—Y, where X is a substituent attached on the N-terminal of a peptide, X can be H, CH₃CO, C₆H₅CO, or C₆H₅CH₂CO; and Y is a substituent attached on the C-terminal of a peptide with the following general structure:

C(α)-CO—Y, wherein Y can be OH, NH₂, OCH₃, OCH₂C₆H₅, or NHCH₃ and Y can be from zero to nine amino acids and

R_{1d} is Ile, Leu, Val, or Phe;

R_{2d} is Gly, Ala;

R_{3d} is Ala, Gly;

R_{4d} is Ser, Thr, or Tyr;

R_{5d} is Trp, Phe, or Tyr;

R_{6d} is His, Lys, Arg, or Tyr;

R_{7d} is Arg, His, or Lys;

R_{8d} is Pro, Leu, or Val;

R_{9d} is Asp, Glu, Arg, or Lys;

R_{10d} is Lys, Arg, or His;

R_{11d} is Ala, Cys, or Ser;

R_{12d} is Ala, Cys, or Ser;

R_{13d} is Ile, Leu, or Phe;

R_{14d} is Gly, Ala;

R_{15d} is Tyr, Thr, Ser;

R_{16d} is Gln, Asn, Arg, or Lys;

R_{17d} is Lys, Arg, or His;

R_{18d} is Arg, His, or Lys;

R_{19d} is Pro, Leu, or Val;

R_{20d} is Ile, Leu, or Val;

R_{21d} is Pro, Leu, or Val;

and where,

if R_{11d} is Cys then R_{12d} can be Cys, penicillamine or tertiary butyloxycarbonyl-a-aminobutyric acid;

if R_{12d} is Cys then R_{11d} can be Cys, penicillamine, or tertiary butyloxycarbonyl-a-aminobutyric acid; and,

R_{11d} and R_{12d} can be penicillamine, or tertiary butyloxycarbonyl-a-aminobutyric acid;

and, R_{11d} and R_{12d} can be Ala.

It is a further object of the present invention for the preferred embodiment of the D-amino acid containing peptide to have the following formula:

X can be H, CH₃CO; Y can be OH, or NH₂; and, R_{1d} is Leu, R_{2d} is Gly, R_{3d} is Ala, R_{4d} is Ser, R_{5d} is Trp, R_{6d} is His, R_{7d} is Arg, R_{8d} is Pro, R_{9d} is Asp, R_{10d} is Lys, R_{11d} is Ala, R_{12d} is Cys, R_{13d} is Leu, R_{14d} is Gly, R_{15d} is Tyr, R_{16d} is Gln, R_{17d} is Lys, R_{18d} is Arg, R_{19d} is Pro, R_{20d} is Leu, R_{21d} is Pro.

A most preferred embodiment of the D-amino acid peptide is:

X is H, Y is NH₂; and, R_{1d} is Leu, R_{2d} is Gly, R_{3d} is Ala, R_{4d} is Ser, R_{5d} is Trp, R_{6d} is His, R_{7d} is Arg, R_{8d} is Pro, R_{9d} is Asp, R_{10d} is Lys, R_{11d} is Ala, R_{12d} is Cys, R_{13d} is Leu, R_{14d} is Gly, R_{15d} is Tyr, R_{16d} is Gln, R_{17d} is Lys, R_{18d} is Arg, R_{19d} is Pro, R_{20d} is Leu, R_{21d} is Pro.

It is another object of the present invention for the preferred C-terminal truncation peptide of the D-amino acid peptide to have at least the following fragment:

X—R_{1d}—R_{2d}—R_{3d}—R_{4d}—R_{5d}—R_{6d}—R_{7d}—R_{8d}—Y

and where;

R_{1d} is Ile, Leu, or Phe;

R_{2d} is Gly, Ala, or Val;

R_{3d} is Ala, Val, or Gly;

R_{4d} is Ser, Thr, or Tyr;

R_{5d} is Trp, Phe, Tyr, or Leu;

R_{6d} is His, Lys, Arg, or Trp;

R_{7d} is Arg, His, or Lys;

R_{8d} is Pro, Leu, or Val.

In a further preferred embodiment the C-terminal truncation peptide has at least the following fragment;

X is H, Y is NH₂; and, R_{1d} is Leu, R_{2d} is Gly, R_{3d} is Ala, R_{4d} is Ser, R_{5d} is Trp, R_{6d} is His, R_{7d} is Arg, R_{8d} is Pro, R_{9d} is Asp, R_{10d} is Lys. It is another object of the present invention for the D-amino acid peptide to have between 3-30 amino acids, preferably 8-21 amino acids.

It is another object of the invention for the peptide to have a reversed form of said formula,

X—R₂₁—R₂₀—R₁₉—R₁₈—R₁₇—R₁₆—R₁₅—R₁₄—R₁₃—R₁₂—R₁₁—R₁₀—R₉—R₈—R₇—R₆—R₅—R₄—R₃—R₂—R₁—Y

where the amino acids are in an L form or as naturally occurring amino acid.

The preferred embodiment of the reversed form of the peptide is: X can be H, or CH₃CO; Y can be OH, or NH₂; and, R₁ is Leu, R₂ is Gly, R₃ is Ala, R₄ is Ser, R₅ is Trp, R₆ is His, R₇ is Arg, R₈ is Pro, R₉ is Asp, R₁₀ is Lys, R₁₁ is Cys, R₁₂ is Cys, R₁₃ is Leu, R₁₄ is Gly, R₁₅ is Tyr, R₁₆ is Gln, R₁₇ is Lys, R₁₈ is Arg, R₁₉ is Pro, R₂₀ is Leu, R₂₁ is Pro.

The most preferred embodiment of the reversed form of the peptide is: X is H, Y is NH₂; and, R₁ is Leu, R₂ is Gly, R₃ is Ala, R₄ is Ser, R₅ is Trp, R₆ is His, R₇ is Arg, R₈ is Pro, R₉ is Asp, R₁₀ is Lys, R₁₁ is Cys, R₁₂ is Cys, R₁₃ is Leu, R₁₄ is Gly, R₁₅ is Tyr, R₁₆ is Gln, R₁₇ is Lys, R₁₈ is Arg, R₁₉ is Pro, R₂₀ is Leu, R₂₁ is Pro.

It is another object of the present invention for the reversed form of the peptide to have a C-terminal truncation peptide containing at least the following fragment:

X—R₁—R₂—R₃—R₄—R₅—R₆—R₇—R₈—Y,

and wherein;

R₁ is Ile, Leu, or Phe;

R₂ is Gly, Ala, or Val;

R₃ is Ala, Val, or Gly;

R₄ is Ser, Thr, or Tyr;

R₅ is Trp, Phe, Tyr, or Leu;

R₆ is His, Lys, Arg, or Trp;

R₇ is Arg, His, or Lys;

R₈ is Pro, Leu, or Val.

and, a C-terminal truncation peptide preferably containing at least a following fragment, wherein X is H, Y is NH₂; and, R₁ is Leu, R₂ is Gly, R₃ is Ala, R₄ is Ser, R₅ is Trp, R₆ is His, R₇ is Arg, R₈ is Pro, R₉ is Asp, R₁₀ is Lys.

It is another object of the present invention for the reversed form of the peptide to be between 3-30 amino acids, preferably 8-21 amino acids.

It is another object of the present invention for the peptide to be a reversed form of the peptide with D-amino acids, having the formula:

X—R_{21d}—R_{20d}—R_{19d}—R_{18d}—R_{17d}—R_{16d}—R_{15d}—R_{14d}—R_{13d}—R_{12d}—R_{11d}—R_{10d}—R_{9d}—R_{8d}—R_{7d}—R_{6d}—R_{5d}—R_{4d}—R_{3d}—R_{2d}—R_{2d}—Y, wherein an amino acid is in a D form or as an unnaturally occurring amino acid. A preferred embodiment of the reversed formula with D-amino acids is:

X can be H, CH₃CO; Y can be OH, or NH₂; and, R_{1d} is Leu, R_{2d} is Gly, R_{3d} is Ala, R_{4d} is Ser, R_{5d} is Trp, R_{6d} is His, R_{7d} is Arg, R_{8d} is Pro, R_{9d} is Asp, R_{10d} is Lys, R_{11d} is Ala, R_{12d} is Cys, R_{13d} is Leu, R_{14d} is Gly, R_{15d} is Tyr, R_{16d} is Gln, R_{17d} is Lys, R_{18d} is Arg, R_{19d} is Pro, R_{20d} is Leu, R_{21d} is Pro.

A most preferred embodiment of the reversed formula with D-amino acids is:

X is H, Y is NH₂; and, R_{1d} is Leu, R_{2d} is Gly, R_{3d} is Ala, R_{4d} is Ser, R_{5d} is Trp, R_{6d} is His, R_{7d} is Arg, R_{8d} is Pro, R_{9d} is Asp, R_{10d} is Lys, R_{11d} is Ala, R_{12d} is Cys, R_{13d} is Leu, R_{14d} is Gly, R_{15d} is Tyr, R_{16d} is Gln, R_{17d} is Lys, R_{18d} is Arg, R_{19d} is Pro, R_{20d} is Leu, R_{21d} is Pro.

A preferred C-terminal truncation peptide of the reverse peptide containing D-amino acids is at least the following fragment:

X—R_{1d}—R_{2d}—R_{3d}—R_{4d}—R_{5d}—R_{6d}—R_{7d}—R_{8d}—Y

and where,

R_{1d} is Ile, Leu, or Phe;

R_{2d} is Gly, Ala, or Val;

R_{3d} is Ala, Val, or Gly;

R_{4d} is Ser, Thr, or Tyr;

R_{5d} is Trp, Phe, Tyr, or Leu;

R_{6d} is His, Lys, Arg, or Trp;

R_{7d} is Arg, His, or Lys;

R_{8d} is Pro, Leu, or Val.

A preferred embodiment of the the reverse peptide containing D-amino acids is at least the following fragment;

X is H, Y is NH₂; and, R_{1d} is Leu, R_{2d} is Gly, R_{3d} is Ala, R_{4d} is Ser, R_{5d} is Trp, R_{6d} is His, R_{7d} is Arg, R_{8d} is Pro, R_{9d} is Asp, R_{10d} is Lys. It is another object of the present invention for the reverse form of the between 3-30 amino acids, preferably 8-21 amino acids.

It is a further object of the invention for a pharmaceutical composition to be a pharmaceutically acceptable carrier and any one of the peptides or peptide fragments of the present invention. It is another object of the invention that a method of inhibiting entry of HIV-1 into CXCR4-expressing cells involve contacting cells with any one of the peptides or peptide fragments of the invention.

It is a further object of the invention that a method of treating infection by HIV-1, involves administering to an individual an effective amount of any one of the peptides or peptide fragments of the invention.

It is another object of the present invention that a method of inhibiting a disease, a causative agent of the disease requiring entry into CXCR4-expressing cells via CXCR4, involves contacting the cells with any one of the peptides or peptide fragments of the invention. It is a further object of the invention that a method of treating a disease, a causative agent of the disease requiring entry into CXCR4-expressing cells via CXCR4, involves administering to an individual an effective amount any one of the peptides or peptide fragments of the invention.

Abbreviations

| | |
|---|---|
| vMIP-II: | viral Macrophage Inflammatory Protein-II |
| HIV-1: | Human Immunodeficiency Virus type 1 |
| MIP-1α: | Macrophage Inflammatory Protein 1 α |
| FACS: | Fluorescence Activated Cell Sorter |
| SDF-1: | Stromal cell Derived Factor-1 |
| RANTES: | Regulated upon Activation, Normal T cell Expressed |
| | and Secreted |
| Fmoc: | N-(9-fluorenyl)methoxycarbonyl. |

Amino Acid Abbreviations

The nomenclature used to describe polypeptide compounds of the present invention follows the conventional practice wherein the amino group is presented to the left and the carboxy group to the right of each amino acid residue. In the formulae representing selected specific embodiments of the present invention, the amino-and carboxy-terminal groups, although not specifically shown, will be understood to be in the form they would assume at physiologic pH values, unless otherwise specified. In the amino acid structure formulae, each residue is generally represented by a three-letter designation, corresponding to the trivial name of the amino acid, in accordance with the following schedule:

| | |
|---|---|
| Alanine | Ala |
| Cysteine | Cys |
| Aspartic Acid | Asp |
| Glutamic Acid | Glu |
| Phenylalanine | Phe |
| Glycine | Gly |
| Histidine | His |
| Isoleucine | Ile |
| Lysine | Lys |
| Leucine | Leu |
| Methionine | Met |
| Asparagine | Asn |
| Proline | Pro |
| Glutamine | Gln |
| Arginine | Arg |
| Serine | Ser |
| Threonine | Thr |
| Valine | Val |
| Tryptophan | Trp |
| Tyrosine | Tyr |

Definitions

The following definitions, of terms used throughout the specification, are intended as an aid to understanding the scope and practice of the present invention.

A “peptide” is a compound comprised of amino acid residues covalently linked by peptide bonds.

The expression “amino acid” as used herein is meant to include both natural and synthetic amino acids, and both D and L amino acids. “Natural amino acid” means any of the twenty primary, naturally occurring amino acids which typically form peptides, polypeptides, and proteins. “Synthetic amino acid” means any other amino acid, regardless of whether it is prepared synthetically or derived from a natural source. As used herein, “synthetic amino acid” also encompasses chemically modified amino acids, including but not limited to salts, derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half life without adversely affecting their activity. Additionally, a disulfide linkage may be present or absent in the peptides of the invention, as long as anti-HIV activity is maintained.

Amino acids have the following general structure:

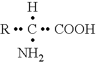

Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group. Peptides comprising a large number of amino acids are sometimes called “polypeptides”. The amino acids of the peptides described herein and in the appended claims are understood to be either D or L amino acids with L amino acids being preferred.

As used herein, “protected” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protected” with respect to a terminal carboxyl group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

By “N-terminal truncation fragment” with respect to an amino acid sequence is meant a fragment obtained from a parent sequence by removing one or more amino acids from the N-terminus thereof.

By “C-terminal truncation fragment” with respect to an amino acid sequence is meant a fragment obtained from a parent sequence by removing one or more amino acids from the C-terminus thereof.

### DESCRIPTION OF THE DRAWINGS

FIG. 1. The CXCR4 binding of peptides, V1 (SEQ. ID. NO: 2) (•), V2 (SEQ. ID. NO: 3) (●), V3 (SEQ. ID. NO: 4) (•) as well as SDF-1α (•) and vMIP-II (◯) as characterized by ¹²⁵I-SDF-1α competitive binding assay. The results shown here are the mean values of three independent assays. Data were processed by using Prism 2.01 (Graphpad Software, Inc., CA). The mean values of three independent experiments are shown.

FIG. 2. Inhibition by vMIP-II derived peptides of HIV-1 coreceptor function of CXCR4 for vSC60 (BH10) T-tropic and 89.6 dual-tropic isolates in a cell-cell fusion assay. The bars represent the mean values of at least three independent assays, whereas the error bars are the standard errors (±S.E.).

FIG. 3. Intracellular calcium influx in sup T1 (a) and CCR5 transfected 293 cells (b). The V1 peptide (SEQ. ID. NO: 2) with indicated concentrations and SDF-1 (100 nM) or MIP-1β (100 nM) were sequentially used to treat sup T1 and 293 cells, respectively.

FIG. 4. Inhibition by the VI peptide (SEQ. ID. NO: 2) of chemotaxis of Sup T1 cells induced by SDF-1. The bars represent the mean values of three independent assays, whereas the error bars are the standard errors (±S.E.).

### DESCRIPTION OF THE INVENTION

Experimental Protocol

Materials—

Recombinant human chemokines SDF-1, MIP-1β and vMIP-II (R & D systems, Minneapolis, Minn.) were lyophilized and dissolved as 1 μg/μl or 2.5 μg/μl stock solutions in sterile phosphate-buffered saline (PBS) and stored at —20° C. in aliquots. The radioiodinated SDF-1α and MIP-1β were purchased from DuPont NEN. The specific activity of ¹²⁵I-SDF-1α and ¹²⁵I-MIP-1β were 2200 Ci/mmol. Cell culture media and G418 were purchased from Life Technologies, Inc. The anti-CXCR4 monoclonal antibody (mAb) 12G5 (Endres, M. J., et al., Cell, 87:745-756, 1996) was purchased from PharMingen (San Diego, Calif.). 293 and NIH/3T3 cells were kindly provided by Robert W. Doms of University of Pennsylvania and maintained in Dulbecco's modified Eagle's medium plus 10% fetal bovine serum. The human pcCXCR4 and recombinant vaccinia viruses encoding two Envs of HIV-1, vSC60 (BH10) (S. Chakrabarti and B. Moss, personal communication) and vBD3 (89.6), and T7 RNA polymerase, vTF1.1 (Alexander, W., et al., J Virol, 66:2934-2942, 1992), were also generous gifts from Robert W. Doms.

Peptide Synthesis

The peptides were prepared by solid phase synthesis using Fmoc-strategy on a 430A peptide synthesizer (Applied Biosystems, Foster City, Calif.) and a 9050 Pepsynthesizer Plus (Perseptive Biosystems, Cambridge, Mass.), as described previously (Satoh, T., et al., J Biol Chem, 272:12175-12180, 1997; Li, S., et al, J Biol Chem, 273:16442-16445, 1998). The side chain protecting groups of N^{•}-Fmoc (N-(9-fluorenyl)methoxycarbonyl) amino acids were: Arg, Pmc; Asp, OtBu; Cys, Trt; Gln, Trt; His, Trt; Lys, Boc; Ser, tBu, Tyr, tBu; and Trp, Boc (Pmc=2,2,5,7,8-pentamethyl-chroman-6-sulfonyl, OtBu=tert-butyl ester, Trt=Trityl, Boc=tert-butyloxycarbonyl and tBu=tert-butyl ester). In every coupling reaction step, a 4-fold excess of N^{•}-Fmoc amino acid, O-benzotriazol-1-yl-N,N,N′,N′-tetramethyluronium hexafluorophosphate, and 1-hydroxybenzotriazole, and 10-fold excess of diisopropylethylamine were used. The cleavage of peptides from the resin was carried out with the cleavage reagent (trifluoroacetic acid:thioanisole:phenol:water:ethandithiol:triisopropylsilane/81.5:5:5:5:2.5:1) for 2 h at room temperature with gentle stirring. Crude peptides were precipitated in ice-cold methyl-t-butyl ether, centrifuged, and lyophilized. The crude peptides were then purified by preparative HPLC using a Dynamax-300 Å C₁₈ 25 cm×21.4 mm I.D. column with two solvent systems of 0.1% TFA/H₂O and 0.1% TFA/acetonitrile. Fractions containing the appropriate peptide were pooled together and lyophilized. The purity of the final product was assessed by analytical reverse phase high performance liquid chromatography, capillary electrophoresis and matrix-assisted laser desorption/ionization time-of-flight mass spectrometry. All peptides were at least 95% pure.

The following peptides were synthesized according to the above procedure:

| | |
|---|---|
| V1 (VMIP-II, 1-21) | LGASWHRPDKCCLGYQKRPLP |
| V2 (VMIP-II, 6-18) | HRPDKCCLGYQKR |
| V3 (VMIP-II, 1-10) | LGASWHRPDK |
| V4 (VMIP-II, 13-34) | LGYQKRPLPQVLLSSWYPTSQL |
| V5 (SDF-1,1-4, vMIP-II, 6-18) | KPVSHRPDKCCLGYQKRPLP |
| V6 (vMIP-II, 22-44) | QVLLSSWYPTSQLCSKPGVIFLT |
| V7 (vMIP-II, 36-57) | SKPGVIFLTKRGRQVCADKSKD |
| V8 (vMIP-II, 51-71) | ADKSKDWVKKLMQQLPVTAR |
| V9 (vMIP-II, 30-40, cyclic-) | CTSQLASKPGC |
| V10 (vMIP-II, 41-51, cyclic-) | CFLTKRGRQVC |
| AV1 (V1 mutant, C11A & C12A) | LGASWHRPDKAALGYQKRPLP |
| V1-1 (V1 mutant, L1A) | AGASWHRPDKCCLGYQKRPLP |
| V1-2 (V1 mutant, W5A) | LGASAHRPDKCCLGYQKRPLP |
| V1-3 (V1 mutant, R7A) | LGASWHAPDKCCLGYQKRPLP |
| V1-4 (V1 mutant, K9A) | LGASWHRPDACCLGYQKRPLP |
| V1-5 (V1 mutant, C11A) | LGASWHRPDKACLGYQKRPLP |
| V1-6 (V1 mutant, Q15A) | LGASWHRPDKCCLGYAKRPLP |
| V1-7 (V1 mutant, R17A) | LGASWHRPDKCCLGYQKAPLP |
| RV1 (vMIP-II, 1-21, reserved) | PLPRKQYGLCCKDPRHWSAGL |
| DV1 (VMIP-II, 1-21, all D-amino acid) | lgaswhrpdkcclgyqkrplp |
| RDV1 (vMIP-II, 1-21, reserved, all D- | plprkqyglcckdprhwsagl |
| amino acid) | |
| V1-DCL (vMIP-II, 1-1O, D-amino scid, 12- | lgaswhrpdkCCLGYQKRPLP |
| 21 L-amino acid) | |
| V1-LCD (vMIP-II, 1-1O, L-amino acid, 11- | LGASWHRPDKCclgyqkrplp |
| 21 D-amino acid) | |
| D1 (DV1 mutant, L1A) | agaswhrpdkcclgyqkrplp |
| D2 (DV1 mutant, W5A) | lgasahrpdkcclgyqkrplp |
| D3 (DV1 mutant, R7A) | lgaswhapdkcclgyqkrplp |
| D4 (DV1 mutant, K9A) | lgaswhrpdacclgyqkrplp |
| D5 (DV1 mutant, C11A) | lgaswhrpdkaclgyqkrplp |
| D6 (DV1 mutant, Q15A) | lgaswhrpdkcclgyakrplp |
| D7 (DV1 mutant, R17A) | lgaswhrpdkcclgyqkaplp |
| AD1 (DV1 mutant, C11A & C12 A) | lgaswhrpdkaalgyqkrplp |
| L-10 (DV1 deletion, 1-10) | lgaswhrpdk |

Upper case represents L-amino acid residues, and lower case represents D-amino acid residues.

Flow Cytometry

Sup T1 cells (2×10⁵) were washed with FACS buffer (0.5% bovine serum albumin, 0.05% sodium azide in PBS) and incubated with an anti-CXCR4 monoclonal antibody (mAb) 12G5 (10 μg/ml) for 30 min at 4° C. After washing with FACS buffer, cells were incubated with 10 μg FITC conjugated goat anti-mouse IgG (Southern Biotechnology Associates, Inc. Birmingham, Ala.) for 30 min at 4° C. After washing twice with FACS buffer, cells were fixed in the fixing buffer (2% palaformaldehyde in PBS) and then analyzed on a FACScan flow cytometer (Coulter EPICS Elite, Coolten Corp., Hialeah, Fla.).

¹²⁵I-SDF-1α Competitive Binding to CXCR4

CEM-T4 cells were harvested and washed twice with PBS. Competition binding experiments were performed using a single concentration (0.2 nM) of ¹²⁵I-SDF-1• in the presence of increasing concentrations of unlabeled ligands in a final volume of 100 μl of binding buffer (50 nM HEPES pH 7.4, 1 nM CaCl₂, 5 nM MgCl₂, 0.1% bovine serum albumin) containing 2×10⁵ cells. Nonspecific binding was determined by the addition of 100 nM unlabeled SDF-1α Samples were incubated for 60 min at room temperature. The incubation was terminated by separating the cells from the binding buffer by centrifugation and washing once with 500 μl of cold binding buffer. Bound ligands were quantitated by counting γ emissions.

¹²⁵I-MIP-1β Competitive Binding to CCR5

Following a similar experimental procedure as described above, 293 cells transfected with CCR5 and ¹²⁵I-MIP-1β were used to determine the specific binding activity of peptides to CCR5.

Gene Reporter Fusion Assay

Following a modified procedure published by our lab (Zhou, N., et al., Eur J Immunol, 30:164-173, 2000) and others (Doranz, B. J., et al., Cell, 85:1149-1158, 1996; Doranz, B. J., et al., J Virol, 71:6305-6314, 1997; Rucker, J., et al., Methods Enzymol, 288:118-133, 1997) a gene reporter fusion assay was used to determine the inhibition of the peptides on coreceptor activity of CXCR4 and CCR5 in mediating HIV-1 viral entry. HIV-1 Env proteins and T7 RNA polymerase were introduced into effector 293 cells by infection with recombinant vaccinia virus and incubated overnight at 32° C. in the presence of rifampicin (100 μg/ml). NIH/3T3 target cells were co-transfected in 6-well plates with plasmids encoding CD4, CXCR4 or CCR5 and luciferase under control of T7 promotor by CaPO₄ transfection and incubated at 37° C. overnight. To initiate fusion, 10⁵ effector cells were added to each well and incubated at 37° C. in the presence of ara-C and rifampicin. After 5 h of fusion, cells were lysed in 150 μl of reporter lysis buffer (Promega) and assayed for luciferase activity by using commercially available reagents (Promega).

Intracellular Calcium

Sup T1 cells and CCR5 transfected 293 cells were used to measure the intracellular calcium influx. [Ca²⁺]ᵢ was measured using excitation at 340 and 380 nm on a fluorescence spectrometer (Perkin Elmer LS50). Calibration was performed using 10% Triton X-100 for total fluorophore release and 0.5 M EGTA to chelate free Ca²⁺. Intracellular Ca²⁺ concentrations were calculated by using the fluorescence spectrometer measurement program.

Chemotaxis

Migration of Sup T1 cells was assessed in disposable Transwell trays (Costar, Cambridge, Mass.) with 6.5 -mm diameter chambers and membrane pore size of 3 μM. SDF-1 at 100 nM (kindly provided by Elias Lolis of Yale University) in 0.5% BSA RPMI 1640 was added to the lower well. 100 μl of Sup T1 cells at 1×10⁷ cells/ml in the same medium without SDF-1 was added to the upper well. For peptide inhibition experiments, the cells were preincubated with various concentrations of the peptide for 15 min at 25° C. The peptide at the same concentration was also added to the lower well. After incubation at 37° C. and 5% CO₂ for 4 h, cells that migrated to the lower well were counted. Chemotactic migration was determined by subtraction of cells migrated in medium alone (Blank control experiment).

Results:

The V1 Peptide Binds CXCR4 but not CCR

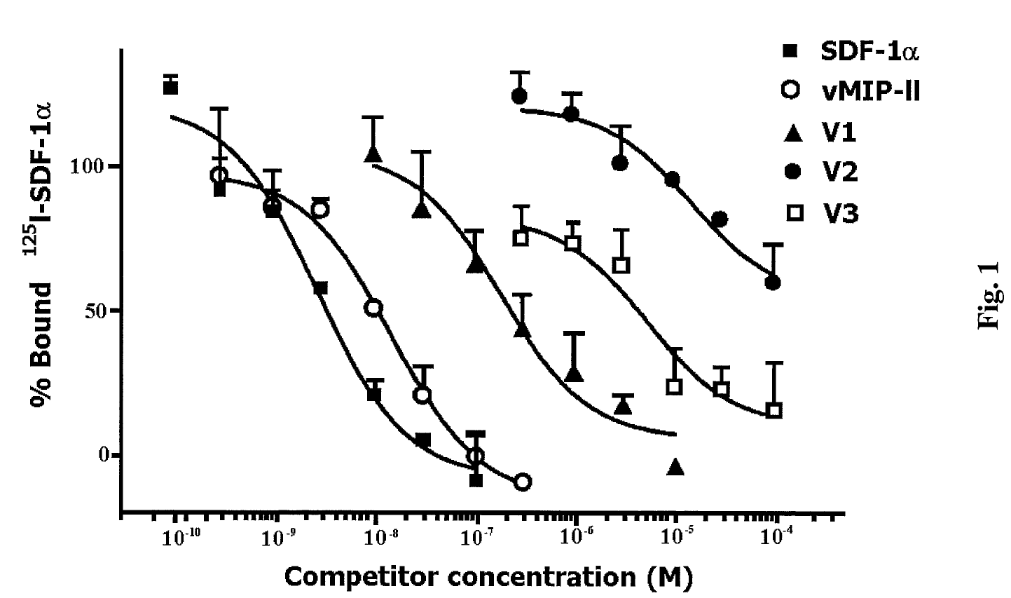

The V1 peptide (SEQ. ID. NO: 2) was synthesized corresponding to residues 1-21 of the N-terminal region of vMIP-II (SEQ. ID. NO: 1) (Table 1). Since vMIP-II interacts with CXCR4 and CCR5 (Kledal, T. N., et al., Science, 277:1656-1659, 1997), we tested the binding activity of V1 peptide (SEQ. ID. NO: 2) with both receptors. For CXCR4 binding, the peptide, together with native vMIP-II and SDF-1α as controls, were examined by using both ¹²⁵I-SDF-1α and anti-CXCR4 mAb 12G5 competitive binding assays (FIG. 1 and Table 2). The V1 peptide (SEQ. ID. NO: 2) strongly competes with the CXCR4 binding of ¹²⁵I-SDF-1α in a concentration dependent manner with an IC₅₀ of 190 nM. Thus, the V1 peptide (SEQ. ID. NO: 2) appears to have much higher CXCR4 binding affinity than other reported peptides derived from SDF-1 N-terminus (Loetscher, P., et al., J Biol Chem, 273:22279-22283, 1998; Heveker, N., et al., Current Biology, 8:369-376, 1998). Since the dimerization of a cysteine-containing peptide derived from SDF-1 N-terminus has been reported to contribute to receptor binding (Loetscher, P., et al., J Biol Chem, 273:22279-22283, 1998), dimer formation of the V1 peptide (SEQ. ID. NO: 2), which contains two cysteines, was examined. Analysis by mass spectrometry demonstrated a pure monomer with no dimer detectable, thereby excluding the contribution of dimerization to the strong CXCR4 binding by the V1 peptide (SEQ. ID. NO: 2).

To further characterize residues within the N-terminus of vMIP-II (SEQ. ID. NO: 1) important for CXCR4 recognition, truncated V1 analogs were synthesized (Table 1). The V2 peptide (residues 6-18 of vMIP-II, SEQ. ID. NO: 3) containing truncation on both ends of V1 (SEQ. ID. NO: 2) showed a significant loss in CXCR4 binding, whereas the V3 peptide (residues 1-10 of vMIP-II, SEQ. ID. NO: 4) containing the first half of V1 sequence (SEQ. ID. NO: 2) retained some activity (FIG. 1). The interaction of these peptides with CCR5 receptor was tested in a competitive binding assay using radiolabeled MIP-1β. These peptides did not show any binding activity with CCR5. These results demonstrated that the peptides derived from the N-terminus of vMIP-II (SEQ. ID. NO: 1) interact with CXCR4 but not CCR5. This is in contrast with native vMIP-II which recognizes both receptors.

The V1 Peptide Selectively Inhibits T- and Dual-Tropic HIV-1 Entry

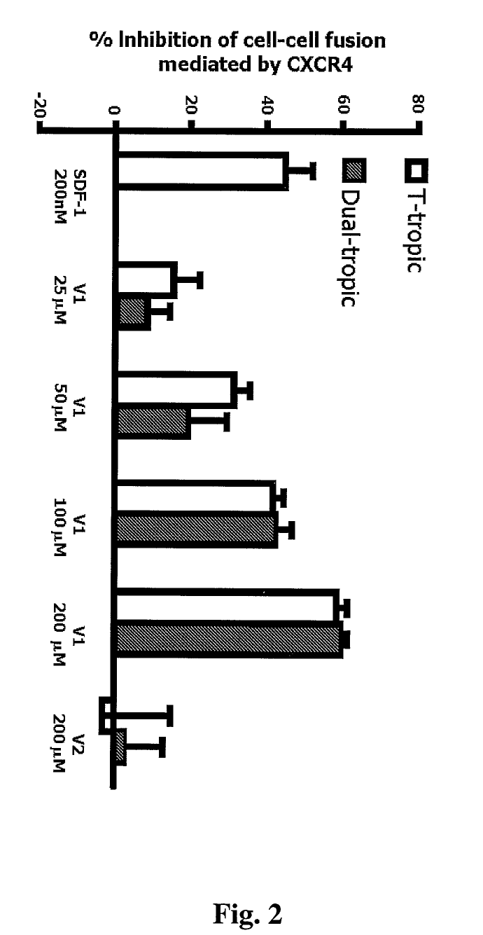

A cell-cell fusion assay was used to determine the ability of CXCR4 and CCR5 peptides in their ability to block the coreceptor function, which mediates cell entry of various HIV-1 isolates. The V1 peptide (SEQ. ID. NO: 2) showed inhibition of both T- and dual-tropic HIV-1 gp120-mediated cell-cell fusion via CXCR4 (FIG. 2). As expected from its significant loss in CXCR4 binding (FIG. 1), the truncated V2 peptide (SEQ. ID. NO: 3) did not show any activity. On the other hand, both V1 (SEQ. ID. NO: 2) and V2 (SEQ. ID. NO: 3) peptides displayed no effect on M-tropic HIV-1 gp120-mediated cell-cell fusion via CCR5. These results were consistent with binding studies and demonstrated that the V1 peptide (SEQ. ID. NO: 2) selectively inhibited CXCR4 coreceptor function in mediating HIV-1 entry.

The V1 Peptide Blocks the Signaling and Chemotaxis of SDF-1 via CXCR4

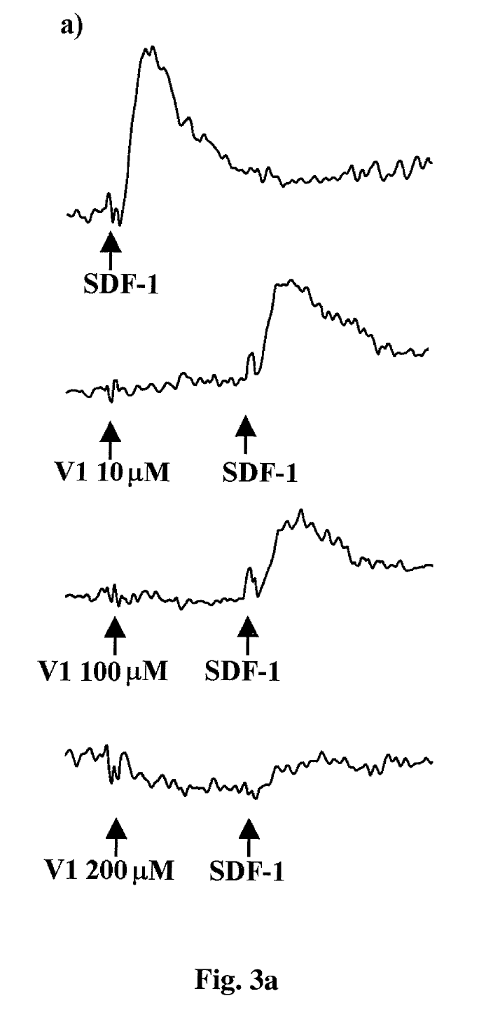

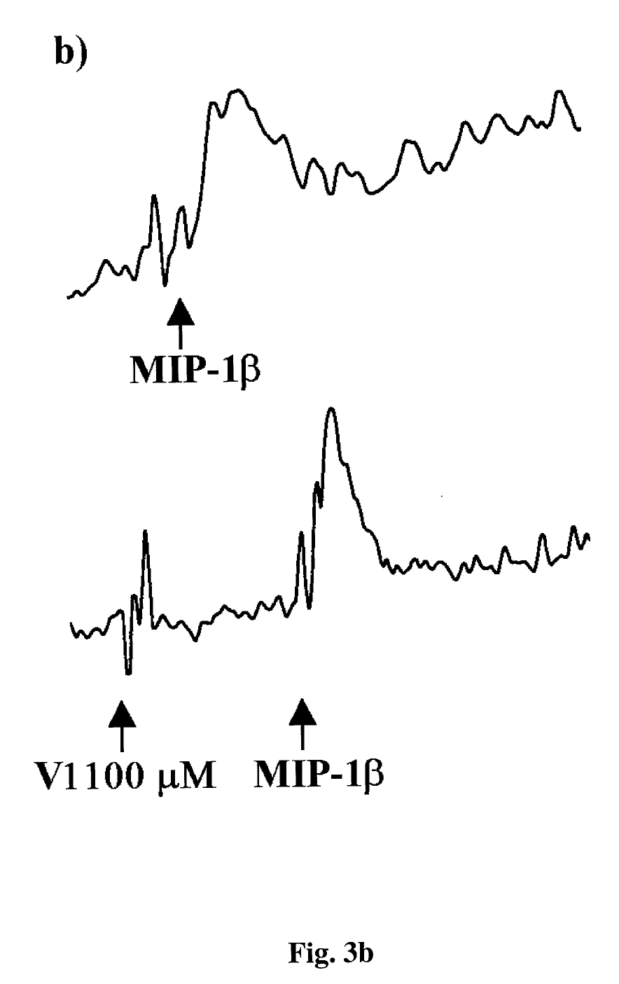

As the V1 peptide (SEQ. ID. NO: 2) can bind CXCR4 receptor, its ability to induce an intracelluar signal or interfere with SDF-1 signaling via CXCR4 was studied by measuring intracellular calcium influx in Sup T1 cells expressing the receptor. At various concentrations, the peptide did not show any signaling activity via CXCR4, thereby revealing it's activity as an antagonist (FIG. 3a). In addition, this peptide interfered with the signaling of SDF-1, a natural CXCR4 ligand, and almost completely blocked SDF-1 signal at the concentration of 200 μM (FIG. 3a).

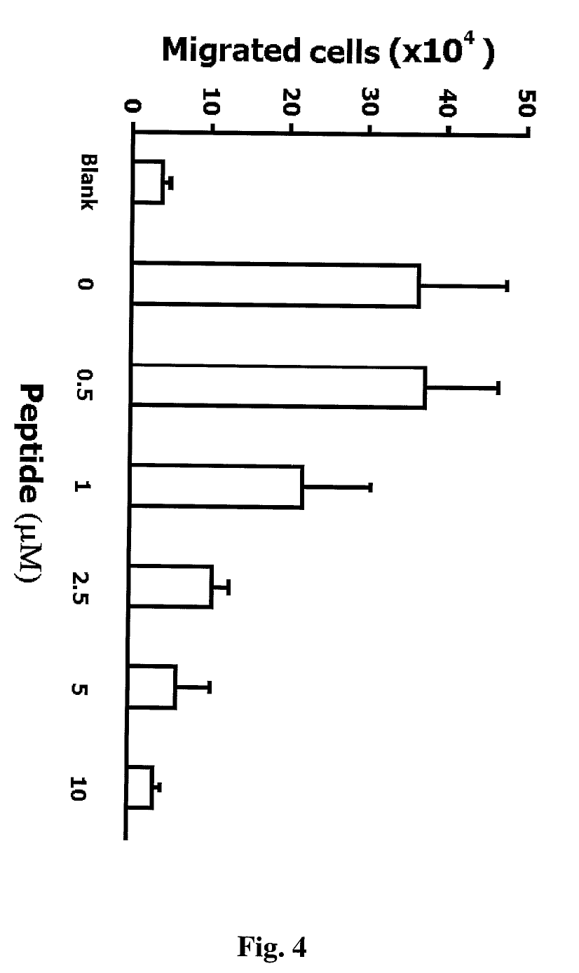

The effect of the V1 peptide (SEQ. ID. NO: 2) on signal transduction via CCR5 was also tested in 293 cells transfected with CCR5. As expected from its lack of binding to CCR5, the peptide neither displayed signaling activity nor blocked the signal induced by MIP-1β via CCR5 (FIG. 3b). The V2 peptide (SEQ. ID. NO: 3), which does not bind CXCR4 or CCR5 (FIG. 1), did not show any effect on CXCR4 or CCR5 signal transduction. In addition to calcium influx, the V1 peptide (SEQ. ID. NO: 2) was tested in assays of chemotaxis of Sup T1 cells. Consistent with its ability to interfere with SDF-1 signaling via CXCR4, the V1 peptide (SEQ. ID. NO: 2) was found to inhibit the chemotactic activity of SDF-1 in a concentration dependent manner (FIG. 4).

Pharmaceutical Compositions

The present invention provides methods for treating HIV-1 infection by inhibiting viral entry into cells expressing the CXCR4 receptor. Such CXCR4-expressing cells include, for example, T-cells. Accordingly, one or more vMIP-II peptides according to the invention is administered to a patient in need of such treatment. A therapeutically effective amount of the drug may be administered as a composition in combination with a pharmaceutically carrier.

Pharmaceutically acceptable carriers include physiologically tolerable or acceptable diluents, excipients, solvents, adjuvants, or vehicles, for parenteral injection, for intranasal or sublingual delivery, for oral administration, for rectal or topical administration or the like. The compositions are preferably sterile and nonpyrogenic. Examples of suitable carriers include but are not limited to water, saline, dextrose, mannitol, lactose, or other sugars, lecithin, albumin, sodium glutamate cysteine hydrochloride, ethanol, polyols (propyleneglycol, ethylene, polyethyleneglycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, agar-agar and tragacanth, or mixtures of these substances, and the like.

The pharmaceutical compositions may also contain minor amounts of nontoxic auxiliary substances such as wetting agents, emulsifying agents, pH buffering agents, antibacterial and antifungal agents (such as parabens, chlorobutanol, phenol, sorbic acid, and the like). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions.

Compositions containing the vMIP-II peptides are administered by any convenient route which will result in delivery to the site of infection of CXCR4-expressing cells by HIV-1, in an amount effective for inhibiting that infection from proceeding. Modes of administration include, for example, orally, rectally, parenterally (intravenously, intramuscularly, intraarterially, or subcutaneously), intracisternally, intravaginally, intraperitoneally, locally (powders, ointments or drops), or as a buccalor nasal spray or aerosol.

The pharmaceutical compositions are most effectively administered parenterally, preferably intravenously or subcutaneously. For intravenous administration, they may be dissolved in any appropriate intravenous delivery vehicle containing physiologically compatible substances, such as sodium chloride, glycine, and the like, having a buffered pH compatible with physiologic conditions. Such intravenous delivery vehicles are known to those skilled in the art. In a preferred embodiment, the vehicle is a sterile saline solution. If the peptides are sufficiently small, other preferred routes of administration are intranasal, sublingual, and the like. Intravenous or subcutaneous administration may comprise, for example, injection or infusion.

The vMIP-II-derived peptides according to the invention can be administered in any circumstance in which inhibition of HIV infection is desired. The peptides of the invention may be used for treatment of subjects as a preventative measure to avoid HIV infection, or as a therapeutic to treat patients already infected with HIV. The viruses whose transmission may be inhibited by the peptides of the invention include strains of HIV-1, but is most useful for those strains which gain entry via the CXCR4, such as T-tropic and dual-tropic strains. T-tropic strains utilize CXCR4 for entry, while dual-tropic strains utilize CXCR4 or CCR5 (Simmons et al., J. Virol. 70:8355-60, 1996). The peptides of the invention may be used prophylactically in uninfected individuals after exposed to an HIV virus. Examples of such uses include in the prevention of viral transmission from mother to infant, and following accidents in healthcare wherein workers may become exposed to HIV-contaminated blood, syringes and the like. The peptides may be administered to other individuals at risk of contracting HIV, such as homosexuals, prostitutes and intravenous drug users.

The vMIP-II -derived peptides may be administered alone or in combination with other peptides or other anti-HIV pharmaceutical agents. The effective amount and method of administration will vary based upon the sex, age, weight and disease stage of the patient, whether the administration is therapeutic or prophylactic, and other factors apparent to those skilled in the art. Based upon the studies described herein, a suitable dosage of peptide is a dosage which will attain a tissue concentration of from about 1 to about 100 •M, more preferably from about 10 to about 50 •M, most preferably about 25 •M. It is contemplated that lower or higher concentrations would also be effective. The tissue concentration may be derived from peptide blood levels.

The amount of active agent administered depends upon the degree of the infection. Those skilled in the art will derive appropriate dosages and schedules of administration to suit the specific circumstances and needs of the patient. Doses are contemplated on the order of from about 0.01 to about 1, preferably from about 0.1 to about 0.5, mg/kg of body weight. The active agent may be administered by injection daily, over a course of therapy lasting two to three weeks, for example. Alternatively, the agent may be administered by continuous infusion, such as via an implanted subcutaneous pumps.

Discussion:

The viral chemokine vMIP-II differs from all known human chemokines in that vMIP-II binds with high affinity to a number of both CC and CXC chemokine receptors (Kledal, T. N., et al., Science, 277:1656-1659, 1997). This unique property of vMIP-II presents an intriguing avenue to probe the structural basis for the promiscuous receptor interaction. The present invention relates to determining if the common binding sites of vMIP-II have been optimized by the virus for multiple receptor interactions, or if distinctive binding determinants have evolved for different receptors.

A synthetic peptide approach was used to study the role of the N-terminus of vMIP-II (SEQ. ID. NO: 1) in the recognition with two important chemokine receptors, CXCR4 and CCR5. The N-terminal region is most diverse among vMIP-II and other chemokines and, on the basis of the importance of N-termini in other chemokines (Clark-Lewis, I., et al., J Leuk Biol, 57:703-11, 1995), critical for the unique function of vMIP-II. The V1 peptide (SEQ. ID. NO: 2) of the present invention corresponds to this region and is shown to interact with CXCR4, thereby blocking signal transduction and the coreceptor function that mediates HIV-1 entry. This indicates that the N-terminus of vMIP-II is essential for its biological function through CXCR4. In contrast to its potent activities via CXCR4, V1 peptide (SEQ. ID. NO: 2) did not display any interaction with CCR5 or inhibition of CCR5 signaling and coreceptor function. Whereas the native vMIP-II (SEQ. ID. NO: 1) binds and blocks the function of both receptors, the lack of interaction of the N-terminal fragment of vMIP-II with CCR5 implies that other domains, yet to be identified, mediate vMIP-II function via CCR5. Alternatively, the peptide without the other domains of the vMIP-II protein may fail to adopt conformations necessary for CCR5 recognition. However, this is unlikely given the strong interaction of this peptide with another receptor, CXCR4, implying the peptide has the proper structural elements for receptor binding. Taken together, the present invention describes distinctive determinants in vMIP-II (SEQ. ID. NO: 1) that mediate biological function via different receptors.

The important feature of the N-terminus of vMIP-II for CXCR4 recognition was further analyzed with truncated peptide analogs of V1. It has been suggested that a spatial cluster of positive residues in SDF-1 is critical for forming favorable electrostatic interaction with the negative charge surface of the extracellular domains of CXCR4 (Dealwis, C., et al., Proc Natl Acad Sci USA, 95:6941-6946, 1998). A high positive charge is seen in several peptide and nonpeptide inhibitors of CXCR4, such as T22 (Murakami, T., et al., J Exp Med, 186:1389-1393, 1997), ALX40-4C (Doranz, B. J., et al., J Exp Med, 186:1395-1400, 1997), and AMD3100 (Schols, D. et al., J Exp Med, 186:1383-1388, 1997). Interestingly, vMIP-II (SEQ. ID. NO: 1) has a high net positive charge like SDF-1, despite the very low sequence homology between them. Since the V1 peptide (SEQ. ID. NO: 2) derived from the N-terminus of vMIP-II also contains a number of positive charge residues, this raised the question whether these residues play a role in receptor interaction. The V2 peptide (SEQ. ID. NO: 3) of the present invention, which retains all positive residues in the core region of the V1 peptide (SEQ. ID. NO: 2), tested the function of these positively charged residues. The loss of activity in the V2 peptide (SEQ. ID. NO: 3) argued against a primary role of the positive residues in receptor binding. Alternatively, the first five residues of vMIP-II are more critical and their removal in the V2 peptide (SEQ. ID. NO: 3) explains the loss of activity. This is consistent with observations made for other chemokines where the first several residues at the N-terminus are most important for biological function (Heveker, N., et al., Current Biology, 8:369-376, 1998; Hèbert, C. A., et al., J Biol Chem, 266:18989-18994, 1991; Crump, M. P., et al., EMBO Journal, 16:6996-7007, 1997). The role of the first five residues of the N-terminus of vMIP-II was further demonstrated by V3 (SEQ. ID. NO: 4), a shorten analog containing only the N-terminal half of the V1 peptide (SEQ. ID. NO: 2), which retained some activity in CXCR4 binding (FIG. 1).

The V1 peptide (SEQ. ID. NO: 2) of the present invention is a promising lead compound for the development of high affinity ligands for CXCR4. Although a direct comparison with other chemokine derived peptides can not be made due to the difference in binding assay protocols, the relative affinity of the V1 peptide (SEQ. ID. NO: 2) as compared with other CXCR4 binding peptides is estimated by comparing these peptides with native SDF-1. In the present invention, the V1 peptide (SEQ. ID. NO: 2) was shown to compete with the CXCR4 binding of anti-CXCR4 mAb 12G5 and ¹²⁵I-SDF-1α, with IC₅₀ values of 640 and 190 nM, respectively (Table 2 and FIG. 1), which are about 33- and 70-fold less potent than SDF-1, respectively. This compares favorably with other reported peptides derived from the N-terminus of SDF-1 which are about 82- to 1000-fold less potent than SDF-1 (Loetscher, P., et al., J Biol Chem, 273:22279-22283, 1998; Heveker, N., et al., Current Biology, 8:369-376, 1998). In addition to its relatively high CXCR4 affinity among chemokine derived peptides reported so far, the V1 peptide (SEQ. ID. NO: 2) possesses other interesting biological properties, such as the induction of CXCR4 internalization. The potency of the V1 peptide (SEQ. ID. NO: 2) in the cell-cell fusion assay (FIG. 2) was much lower than that in the competition binding assay (FIG. 1 and Table 2). A similar discrepancy in potency between these two assays was also observed for SDF-1, which showed an IC₅₀ of 2.7 nM in ¹²⁵I-SDF-1α competitive binding assay (FIG. 1 and Table 2) but had only 45% inhibition of cell-cell fusion even at 200 nM (FIG. 2). These are due to the relative insensitivity of the cell-cell fusion assay for the quantitative determination of the potency of anti-HIV agents, as previously reported by others (Rucker, J., et al, Methods Enzymol, 288:118-133, 1997). Therefore, the activity of the V1 peptide (SEQ. ID. NO: 2), as well as the control SDF-1, are underestimated in the cell-cell fusion assay. Compared to the cell-cell fusion assay, the inhibitory activity of the V1 peptide (SEQ. ID. NO: 2) in the chemotaxis assay was much higher with an IC₅₀ of about 1 μM, which was more consistent with its CXCR4 binding potency (FIG. 4).

In summary, the characterization of precise binding sites within vMIP-II (SEQ. ID. NO: 1) for CXCR4 and CCR5 is a critical step toward understanding the molecular mechanism of vMIP-II function and development of broad-spectrum HIV inhibitors. The present invention identifies the N-terminus, particularly the first five residues, of vMIP-II as an important binding site for CXCR4. A synthetic peptide derived from this region displays widely different interactions with CXCR4 and CCR5, thus providing experimental support for the notion that distinctive sites within vMIP-II (SEQ. ID. NO: 1) mediate interactions with different chemokine receptors. With its high CXCR4 receptor binding affinity and potent antagonistic effects, the vMIP-II derived peptide of the present invention (V1, SEQ. ID. NO: 2) is good lead compound for the further development of novel small molecular agents that prevent the cellular entry of HIV via CXCR4 coreceptor.

What is claimed is:

1. A peptide fragment of a viral Macrophage Inflammatory Protein-II (vMIP-II) (SEQ. ID. NO: 1), wherein said fragment selectively prevents CXCR4 signal transduction and coreceptor function in mediating an entry of an HIV-1.

2. The peptide fragment of claim 1, wherein said fragment comprises an amino-terminal end of said vMIP-II.

3. The peptide fragment of claim 2, wherein said amino-terminal end comprises amino acid residues 1-21 (V1, SEQ ID NO: 2), or any subfragments therein.

4. The peptide fragment of claim 1, wherein said fragment is a lead compound for development of novel small molecular agents to prevent HIV-1 from entering a cell.

5. A peptide of the formula
X—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—Y
wherein:
X is a substituent attached on the N-terminal of a peptide, X can be H, CH₃CO, C₆H₅CO, or C₆H₅CH₂CO;
Y is a substituent attached on the C-terminal of a peptide with the following general structure,
C(α)-CO—Y
Y can be OH, NH₂, OCH₃, OCH₂C₆H₅, or NHCH₃; Y can be from zero to nine amino acids,
R₁ is Ile, Leu, Val, or Phe;
R₂ is Gly, Ala;
R₃ is Ala, Gly;
R₄ is Ser, Thr, or Tyr;
R₅ is Trp, Phe, Tyr;
R₆ is His, Lys, Arg, or Tyr;
R₇ is Arg, His, or Lys;
R₈ is Pro, Leu, or Val;
R₉ is Asp, Glu, Arg, or Lys;
R₁₀ is Lys, Arg, or His;
R₁₁ is Cys, Ser, or Ala;
R₁₂ is Cys, Ser, or Ala;
R₁₃ is Ile, Leu, or Val;
R₁₄ is Gly, Ala;
R₁₅ is Tyr, Thr, Ser;
R₁₆ is Gln, Asn, Arg, or Lys;
R₁₇ is Lys, Arg, or His;
R₁₈ is Arg, His, or Lys;
R₁₉ is Pro, Leu, or Val;
R₂₀ is Ile, Leu, or Val;
R₂₁ is Pro, Leu, or Val;
and if R₁₁ is Cys then R₁₂ can be Cys, penicillamine or tertiary butyloxycarbonyl-a-aminobutyric acid;
if R₁₂ is Cys then R₁₁ can be Cys, penicillamine, tertiary butyloxycarbonyl-a-aminobutyric acid, and,
R₁₁ and R₁₂ can be penicillamine, or tertiary butyloxycarbonyl-a-aminobutyric acid;
and, R₁₁ and R₁₂ can be Ala.

6. The peptide of claim 5, wherein a preferred embodiment, comprises
X can be H, or CH₃CO; Y can be OH, or NH₂; and, R₁ is Leu, R₂ is Gly, R₃ is Ala, R₄ is Ser, R₅ is Trp, R₆ is His, R₇ is Arg, R₈ is Pro, R₉ is Asp, R₁₀ is Lys, R₁₁ is Cys, R₁₂ is Cys, R₁₃ is Leu, R₁₄ is Gly, R₁₅ is Tyr, R₁₆ is Gln, R₁₇ is Lys, R₁₈ is Arg, R₁₉ is Pro, R₂₀ is Leu, R₂₁ is Pro.

7. The peptide of claim 5, wherein a most preferred embodiment, comprises X is H, Y is NH₂; and, R₁ is Leu, R₂ is Gly, R₃ is Ala, R₄ is Ser, R₅ is Trp, R₆ is His, R₇ is Arg, R₈ is Pro, R₉ is Asp, R₁₀ is Lys, R₁₁ is Cys, R₁₂ is Cys, R₁₃ is Leu, R₁₄ is Gly, R₁₅ is Tyr, R₁₆ is Gln, R₁₇ is Lys, R₁₈ is Arg, R₁₉ is Pro, R₂₀ is Leu, R₂₁ is Pro.

8. The peptide of claim 5, wherein a preferred embodiment comprises a C-terminal truncation peptide contaiings at least the following fragment:
X—R₁—R₂—R₃—R₄—R₅—R₆—R₇—R₈—Y,
and wherein;
R₁ is Ile, Leu, or Phe;
R₂ is Gly, Ala, or Val;
R₃ is Ala, Val, or Gly;
R₄ is Ser, Thr, or Tyr;
R₅ is Trp, Phe, Tyr, or Leu;
R₆ is His, Lys, Arg, or Trp;
R₇ is Arg, His, or Lys;
R₈ is Pro, Leu, or Val.
and, a C-terminal truncation peptide preferably containing at least a following fragment, wherein X is H, Y is NH₂; and, R₁ is Leu, R₂ is Gly, R₃ is Ala, R₄ is Ser, R₅ is Trp, R₆ is His, R₇ is Arg, R₈ is Pro, R₉ is Asp, R₁₀ is Lys.

9. The peptide of claim 1, wherein said peptide comprises between 3-30 amino acids, preferably 8-21 amino acids.

10. A synthetic peptide, wherein each amino acid of said synthetic peptide is a D amino acid, having the formula:
X—R_{1d}—R_{2d}—R_{3d}—R_{4d}—R_{5d}—R_{6d}—R_{7d}—R_{8d}—R_{9d}—R_{10d}—R_{11d}—R_{12d}—R_{13d}—R_{14d}—R_{15d}—R_{16d}—R_{17d}—R_{18d}—R_{19d}—R_{20d}—R_{21d}—Y,
wherein,
X is a substituent attached on the N-terminal of a peptide, X can be H, CH₃CO, C₆H₅CO, or C₆H₅CH₂CO; and
Y is a substituent attached on the C-terminal of a peptide with the following general structure:
C(α)-CO—Y, wherein Y can be OH, NH₂, OCH₃, OCH₂C₆H₅, or NHCH₃ and Y can be from zero to nine amino acids.
R_{1d} is Ile, Leu, Val, or Phe;
R_{2d} is Gly, Ala;
R_{3d} is Ala, Gly;
R_{4d} is Ser, Thr, or Tyr;
R_{5d} is Trp, Phe, or Tyr;
R_{6d} is His, Lys, Arg, or Tyr;
R_{7d} is Arg, His, or Lys;
R_{8d} is Pro, Leu, or Val;
R_{9d} is Asp, Glu, Arg, or Lys;
R_{10d} is Lys, Arg, or His;
R_{11d} is Ala, Cys, or Ser;
R_{12d} is Ala, Cys, or Ser;
R_{13d} is Ile, Leu, or Phe;
R_{14d} is Gly, Ala;
R_{15d} is Tyr, Thr, Ser;
R_{16d} is Gln, Asn, Arg, or Lys;
R_{17d} is Lys, Arg, or His;
R_{18d} is Arg, His, or Lys;
R_{19d} is Pro, Leu, or Val;
R_{20d} is Ile, Leu, or Val;
R_{21d} is Pro, Leu, or Val;
and wherein:
if R_{11d} is Cys then R_{12d} can be Cys, penicillamine or tertiary butyloxycarbonyl-a-aminobutyric acid;
if R_{12d} is Cys then R_{11d} can be Cys, penicillamine, or tertiary butyloxycarbonyl-a-aminobutyric acid;
and,
R_{11d} and R_{12d} can be penicillamine, or tertiary butyloxycarbonyl-a-aminobutyric acid;
and, R_{11d} and R_{12d} can be Ala.

11. The peptide of claim 10, wherein a preferred embodiment comprises the following formula:
X can be H, CH₃CO; Y can be OH, or NH₂; and, R_{1d} is Leu, R_{2d} is Gly, R_{3d} is Ala, R_{4d} is Ser, R_{5d} is Trp, R_{6d} is His, R_{7d} is Arg, R_{8d} is Pro, R_{9d} is Asp, R_{10d} is Lys, R_{11d} is Ala, R_{12d} is Cys, R_{13d} is Leu, R_{14d} is Gly, R_{15d} is Tyr, R_{16d} is Gln, R_{17d} is Lys, R_{18d} is Arg, R_{19d} is Pro, R_{20d} is Leu, R_{21d} is Pro.

12. The peptide of claim 10, wherein a most preferred embodiment comprises the following formula:
X is H, Y is NH₂; and, R_{1d} is Leu, R_{2d} is Gly, R_{3d} is Ala, R_{4d} is Ser, R_{5d} is Trp, R_{6d} is His, R_{7d} is Arg, R_{8d} is Pro, R_{9d} is Asp, R_{10d} is Lys, R_{11d} is Ala, R_{12d} is Cys, R_{13d} is Leu, R_{14d} is Gly, R_{15d} is Tyr, R_{16d} is Gin, R_{17d} is Lys, R_{18d} is Arg, R_{19d} is Pro, R_{20d} is Leu, R_{21d} is Pro.

13. The peptide of claim 10, wherein a preferred C-terminal truncation peptide comprising at least the following fragment:
X—R_{1d}—R_{2d}—R_{3d}—R_{4d}—R_{5d}—R_{6d}—R_{7d}—R_{8d}—Y
and wherein;
R_{1d} is Ile, Leu, or Phe;
R_{2d} is Gly, Ala, or Val;
R_{3d} is Ala, Val, or Gly;
R_{4d} is Ser, Thr, or Tyr;
R_{5d} is Trp, Phe, Tyr, or Leu;
R_{6d} is His, Lys, Arg, or Trp;
R_{7d} is Arg, His, or Lys;
R_{8d} is Pro, Leu, or Val.

14. The peptide of claim 10, wherein a more preferably C-terminal truncation peptide comprises at least the following fragment;
X is H, Y is NH₂; and, R_{1d} is Leu, R_{2d} is Gly, R_{3d} is Ala, R_{4d} is Ser, R_{5d} is Trp, R_{6d} is His, R_{7d} is Arg, R_{8d} is Pro, R_{9d} is Asp, R_{10d} is Lys.

15. The peptide of claim 10, comprising between 3-30 amino acids, preferably 8-21 amino acids.

16. The peptide of claim 5, wherein said peptide comprises a reversed form of said formula, comprising,
X—R₂₁—R₂₀—R₁₉—R₁₈—R₁₇—R₁₆—R₁₅—R₁₄—R₁₃—R₁₂—R₁₁—R₁₀—R₉—R₉—R₈—R₇—R₆—R₅—R₄—R₃—R₂—R₁—Y
wherein an amino acid is in an L form or as naturally occurring amino acid.

17. The peptide of claim 16, wherein a preferred embodiment, comprises
X can be H, or CH₃CO; Y can be OH, or NH₂; and, R₁ is Leu, R₂ is Gly, R₃ is Ala, R₄ is Ser, R₅ is Trp, R₆ is His, R₇ is Arg, R₈ is Pro, R₉ is Asp, R₁₀ is Lys, R₁₁ is Cys, R₁₂ is Cys, R₁₃ is Leu, R₁₄ is Gly, R₁₅ is Tyr, R₁₆ is Gln, R₁₇ is Lys, R₁₈ is Arg, R₁₉ is Pro, R₂₀ is Leu, R₂₁ is Pro.

18. The peptide of claim 16, wherein a most preferred embodiment, comprises
X is H, Y is NH₂; and, R₁ is Leu, R₂ is Gly, R₃ is Ala, R₄ is Ser, R₅ is Trp, R₆ is His, R₇ is Arg, R₈ is Pro, R₉ is Asp, R₁₀ is Lys, R₁₁ is Cys, R₁₂ is Cys, R₁₃ is Leu, R₁₄ is Gly, R₁₅ is Tyr, R₁₆ is Gln, R₁₇ is Lys, R₁₈ is Arg, R₁₉ is Pro, R₂₀ is Leu, R₂₁ is Pro.

19. The peptide of claim 16, wherein a preferred embodiment comprises a C-terminal truncation peptide contaiings at least the following fragment:
X—R₁—R₂—R₃—R₄—R₅—R₆—R₇—R₈—Y,
and wherein;
R₁ is Ile, Leu, or Phe;
R₂ is Gly, Ala, or Val;
R₃ is Ala, Val, or Gly;
R₄ is Ser, Thr, or Tyr;
R₅ is Trp, Phe, Tyr, or Leu;
R₆ is His, Lys, Arg, or Trp;
R₇ is Arg, His, or Lys;
R₈ is Pro, Leu, or Val.
and, a C-terminal truncation peptide preferably containing at least a following fragment, wherein X is H, Y is NH₂; and, R₁ is Leu, R₂ is Gly, R₃ is Ala, R₄ is Ser, R₅ is Trp, R₆ is His, R₇ is Arg, R₈ is Pro, R₉ is Asp, R₁₀ is Lys.

20. The peptide of claim 16, wherein said peptide comprises between 3-30 amino acids, preferably 8-21 amino acids.

21. The peptide of claim 5, wherein said peptide comprises a reversed form of said formula, comprising
X—R_{21d}—R_{20d}—R_{19d}—R_{18d}—R_{17d}—R_{16d}—R_{15d}—R_{14d}—R_{13d}—R_{12d}—R_{11d}—R_{10d}—R_{9d}—R_{8d}—R_{7d}—R_{6d}—R_{5d}—R_{4d}—R_{3d}—R_{2d}—R_{2d}—Y,
wherein an amino acid is in a D form or as an unnaturally occurring amino acid.

22. The peptide of claim 21, wherein a preferred embodiment comprises the following formula:
X can be H, CH₃CO; Y can be OH, or NH₂; and, R_{1d} is Leu, R_{2d} is Gly, R_{3d} is Ala, R_{4d} is Ser, R_{5d} is Trp, R_{6d} is His, R_{7d} is Arg, R_{8d} is Pro, R_{9d} is Asp, R_{10d} is Lys, R_{11d} is Ala, R_{12d} is Cys, R_{13d} is Leu, R_{14d} is Gly, R_{15d} is Tyr, R_{16d} is Gln, R_{17d} is Lys, R_{18d} is Arg, R_{19d} is Pro, R_{20d} is Leu, R_{21d} is Pro.

23. The peptide of claim 21, wherein a most preferred embodiment comprises the following formula:
X is H, Y is NH₂; and, R_{1d} is Leu, R_{2d} is Gly, R_{3d} is Ala, R_{4d} is Ser, R_{5d} is Trp, R_{6d} is His, R_{7d} is Arg, R_{8d} is Pro, R_{9d} is Asp, R_{10d} is Lys, R_{11d} is Ala, R_{12d} is Cys, R_{13d} is Leu, R_{14d} is Gly, R_{15d} is Tyr, R_{16d} is Gln, R_{17d} is Lys, R_{18d} is Arg, R_{19d} is Pro, R_{20d} is Leu, R_{21d} is Pro.

23. The peptide of claim 21, wherein a preferred C-terminal truncation peptide comprising at least the following fragment:
X—R_{1d}—R_{2d}—R_{3d}—R_{4d}—R_{5d}—R_{6d}—R_{7d}—R_{8d}—Y
and wherein;
R_{1d} is Ile, Leu, or Phe;
R_{2d} is Gly, Ala, or Val;
R_{3d} is Ala, Val, or Gly;
R_{4d} is Ser, Thr, or Tyr;
R_{5d} is Trp, Phe, Tyr, or Leu;
R_{6d} is His, Lys, Arg, or Trp;
R_{7d} is Arg, His, or Lys;
R_{8d} is Pro, Leu, or Val.

24. The peptide of claim 21, wherein a more preferably C-terminal truncation peptide comprises at least the following fragment;
X is H, Y is NH₂; and, R_{1d} is Leu, R_{2d} is Gly, R_{3d} is Ala, R_{4d} is Ser, R_{5d} is Trp, R_{6d} is His, R_{7d} is Arg, R_{8d} is Pro, R_{9d} is Asp, R_{10d} is Lys.

25. The peptide of claim 21, comprising between 3-30 amino acids, preferably 8-21 amino acids.

26. A pharmaceutical composition, comprising a pharmaceutically acceptable carrier and a peptide according to claim 5.

27. A pharmaceutical composition, comprising a pharmaceutically acceptable carrier and a peptide according to claim 10.

28. A pharmaceutical composition, comprising a pharmaceutically acceptable carrier and a peptide according to claim 16.

29. A pharmaceutical composition, comprising a pharmaceutically acceptable carrier and a peptide according to claim 21.

30. A method of inhibiting entry of HIV-1 into CXCR4-expressing cells, comprising contacting said cells with a peptide according to claim 5.

31. A method of inhibiting entry of HIV-1 into CXCR4-expressing cells, comprising contacting said cells with a peptide according to claim 10.

32. A method of inhibiting entry of HIV-1 into CXCR4-expressing cells, comprising contacting said cells with a peptide according to claim 16.

33. A method of inhibiting entry of HIV-1 into CXCR4-expressing cells, comprising contacting said cells with a peptide according to claim 21.

34. A method of treating infection by HIV-1, comprising administering to an individual an effective amount of a peptide according to claim 5.

35. A method of treating infection by HIV-1, comprising administering to an individual an effective amount of a peptide according to claim 10.

36. A method of treating infection by HIV-1, comprising administering to an individual an effective amount of a peptide according to claim 16.

36. A method of treating infection by HIV-1, comprising administering to an individual an effective amount of a peptide according to claim 21.

37. A method of inhibiting a disease, a causative agent of said disease requiring entry into CXCR4-expressing cells via CXCR4, comprising contacting said cells with a peptide according to claim 5.

38. A method of inhibiting a disease, a causative agent of said disease requiring entry into CXCR4-expressing cells via CXCR4, comprising contacting said cells with a peptide according to claim 10.

39. A method of inhibiting a disease, a causative agent of said disease requiring entry into CXCR4-expressing cells via CXCR4, comprising contacting said cells with a peptide according to claim 16.

40. A method of inhibiting a disease, a causative agent of said disease requiring entry into CXCR4-expressing cells via CXCR4, comprising contacting said cells with a peptide according to claim 21.

41. A method of treating a disease, a causative agent of said disease requiring entry into CXCR4-expressing cells via CXCR4, comprising administering to an individual an effective amount of a peptide according to claim 5.

42. A method of treating a disease, a causative agent of said disease requiring entry into CXCR4-expressing cells via CXCR4, comprising administering to an individual an effective amount of a peptide according to claim 10.

43. A method of treating a disease, a causative agent of said disease requiring entry into CXCR4-expressing cells via CXCR4, comprising administering to an individual an effective amount of a peptide according to claim 46.

44. A method of treating a disease, a causative agent of said disease requiring entry into CXCR4-expressing cells via CXCR4, comprising administering to an individual an effective amount of a peptide according to claim 21.
